(19) United States
(12) Patent Application Publication
(10) Pub. No.: US 2025/0288345 A1
(43) Pub. Date: Sep. 18, 2025

(54) SYSTEM AND METHODS FOR DETERMINING PATIENT-SPECIFIC TREATMENT PARAMETERS FOR COOLED RADIOFREQUENCY ABLATION

(71) Applicant: Avent, Inc., Alpharetta, GA (US)

(72) Inventor: Kay Sun, Roswell, GA (US)

(21) Appl. No.: 19/028,302

(22) Filed: Jan. 17, 2025

Related U.S. Application Data
(60) Provisional application No. 63/564,719, filed on Mar. 13, 2024.

(51) Int. Cl. A61B 18/12; A61B 18/00; G16H 50/70

(52) U.S. Cl. CPC A61B 18/1206; G16H 50/70; A61B 2018/00023; A61B 2018/00577

(57) ABSTRACT

A method for determining settings for cooled radiofrequency ablation (CRFA) system includes obtaining historical CRFA data associated with a plurality of patients previously treated using the CRFA system, wherein the historical CRFA data includes patient characteristics, operating parameters of the CRFA system, and treatment outcomes associated with each of the plurality of patients; training an ensemble machine learning model to predict success of CRFA procedures based on the historical CRFA data; determining a first set of operating parameters for the CRFA system using the trained ensemble machine learning model, wherein the first set of operating parameters comprise settings of the CRFA system that are determined to affect the success of CRFA procedures; and determining a value or a range of values for each of the first set of operating parameters for use in CRFA treatment procedures using a decision tree-based model.

### CROSS-REFERENCE TO RELATED APPLICATIONS

This Application claims priority to and the benefit of U.S. Provisional Application No. 63/564,719, titled “SYSTEM AND METHODS FOR DETERMINING PATIENT-SPECIFIC TREATMENT PARAMETERS FOR COOLED RADIOFREQUENCY ABLATION”, filed on Mar. 13, 2024, the content of which is hereby incorporated by reference herein in its entirety.

### BACKGROUND

The present disclosure generally relates to cooled radiofrequency ablation (CRFA) and, in some aspects, to determining control inputs and settings for CRFA therapy that result in more positive treatment outcomes. CRFA is a minimally invasive technique of delivering high-frequency electrical current that has been shown to relieve localized pain in many patients. Generally, the high-frequency current used for such procedures is in the radio frequency (RF) range, e.g., between 100 kHz and 1 GHz and more specifically between 400-600 kHz. RF electrical current is typically delivered from a generator via connected electrodes that are placed in a patient's body, in a region of tissue that contains a neural structure suspected of transmitting pain signals to the brain.

Treatment outcome for pain management procedures is typically measured by patient-reported pain score. Therapy success is often defined as at least 50% pain reduction after treatment. With current protocols for patient parameter selection for CRFA and standard thermoelectric settings used in the genicular nerve ablation (e.g., fixed set desired ablation temperature, time, and temperature ramp rate), the therapy success rates for greater than 50% pain reduction ranged from 35% to 71% (Carlone, Grothaus, Jacobs, & Duncan, 2021; Chen et al., 2020; Davis et al., 2019; McCormick et al., 2017). Despite ablating the same anatomy using the same standard therapy settings, the wide range of success rates indicates that other ablation parameters and individual patient characteristics need to be factored into the treatment delivery for improved and consistent therapy success.

### SUMMARY

One implementation of the present disclosure is a method for determining settings for cooled radiofrequency ablation (CRFA) system, the method including: obtaining historical CRFA data associated with a plurality of patients previously treated using the CRFA system, wherein the historical CRFA data includes patient characteristics, operating parameters of the CRFA system, and treatment outcomes associated with each of the plurality of patients; training an ensemble machine learning model to predict success of CRFA procedures based on the historical CRFA data; determining a first set of operating parameters for the CRFA system using the trained ensemble machine learning model, wherein the first set of operating parameters include settings of the CRFA system that are determined to affect the success of CRFA procedures; and determining a value or a range of values for each of the first set of operating parameters for use in CRFA treatment procedures using a decision tree- based model.

Another implementation of the present disclosure is a system for determining settings for use in cooled radiofrequency ablation (CRFA), the system including: one or more processors; and memory having instructions stored thereon that, when executed by the one or more processors, cause the system to: obtain historical CRFA data associated with a plurality of patients previously treated using a CRFA system, wherein the historical CRFA data includes patient characteristics, operating parameters of the CRFA system, and treatment outcomes associated with each of the plurality of patients; train an ensemble machine learning model to predict success of CRFA procedures based on the historical CRFA data; determine a first set of operating parameters for the CRFA system using the trained ensemble machine learning model, wherein the first set of operating parameters include settings of the CRFA system that are determined to affect the success of CRFA procedures; and determine a value or a range of values for each of the first set of operating parameters for use in CRFA treatment procedures using a decision tree-based model.

Yet another implementation of the present disclosure is a non-transitory computer readable medium having instructions stored thereon that, when executed by one or more processors, cause a device to: obtain historical CRFA data associated with a plurality of patients previously treated using a CRFA system, wherein the historical CRFA data includes patient characteristics, operating parameters of the CRFA system, and treatment outcomes associated with each of the plurality of patients; train an ensemble machine learning model to predict success of CRFA procedures based on the historical CRFA data; determine a first set of operating parameters for the CRFA system using the trained ensemble machine learning model, wherein the first set of operating parameters include settings of the CRFA system that are determined to affect the success of CRFA procedures; and determine a value or a range of values for each of the first set of operating parameters for use in CRFA treatment procedures using a decision tree-based model.

Additional features will be set forth in part in the description which follows or may be learned by practice. The features will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

FIG. 1 is a diagram of an example cooled radiofrequency ablation (CRFA) system, according to some implementations.

FIG. 2 is a diagram of a process for training an ensemble machine learning model to predict the success of CRFA therapy and, in turn, to determine optimized CRFA settings, according to some implementations.

FIG. 3 is a diagram of an example decision tree associated with determining optimized CRFA parameters, according to some implementations.

FIG. 4 is a block diagram of patient parameter generation system for generating patient-specific CRFA parameters, according to some implementations.

FIG. 5 is a flow diagram of a process for training and implementing an ensemble machine learning model to determine patient-specific CRFA parameters, according to some implementations.

FIG. 6 is a flow diagram of a process for generating patient-specific CRFA parameters using a trained ensemble machine learning model, according to some implementations.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Referring generally to the figures, systems and methods of generating and applying patient- specific cooled radiofrequency ablation (CRFA) treatment parameters are shown, according to various implementations. CRFA treatment parameters generally include any of the various settings and/or parameters of a CRFA system that can be controlled during a CRFA procedure. CRFA parameters—or simply, “ablation parameters”—can include, but are not limited to, current, voltage, impedance, power, energy, duration at set temperature, maximum temperature, total ablation time, and the like. As mentioned above, studies have shown that success rates can vary between patients, even when ablating the same anatomy using the same standard therapy settings between the patients. To improve success rates and ensure more consistent results between patients, the disclosed systems and methods relate to developing, training, and implementing models for generating patient-specific ablation parameters.

In particular, historical data—including patient characteristics, ablation parameters, and treatment outcomes—is considered and used to develop a predictive model that can be used to identify the features (e.g., ablation parameters and/or patient characteristics) that are most important in predicting the success of a CRFA procedure. In some implementations, ensemble machine learning is used to first identify a list of features (e.g., ablation parameters and/or patient characteristics) that collectively are of the greatest importance to CRFA therapy success by contributing to a highest “predictiveness” for therapy outcomes. In turn, corresponding threshold levels for each feature can be computed, e.g., using decision trees. In some implementations, the ensemble machine learning model is iteratively updated as new CRFA-related data is obtained. Notably, the ensemble machine learning model can be used to determine treatment parameters for new patients based on their characteristics (e.g., age, gender, BMI, etc.), which can in turn be used to operate a CRFA system.

### CRFA System

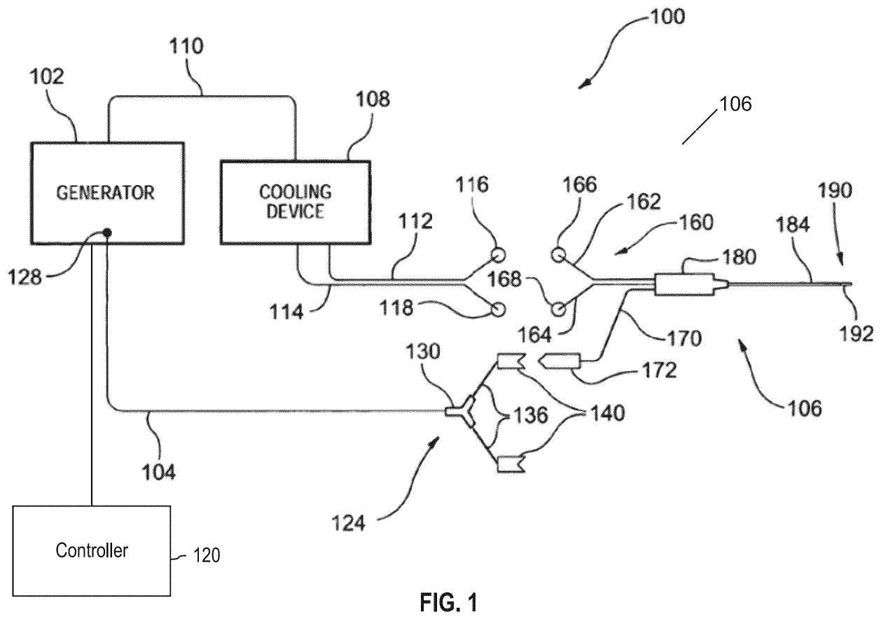

Referring to FIG. 1, a diagram of an example CRFA system 100 is shown, according to some implementations. Specifically, system 100 is an example of a system for applying CRFA to a treatment site on a patient as described herein. System 100 is shown to include a generator 102, a cable 104, one or more probe assemblies (only one probe assembly is shown as probe assembly 106), one or more cooling devices 108 that include a one or more cooling fluid reservoirs and a bidirectional pump assembly (not shown), a pump cable 110, one or more proximal cooling fluid supply tubes 112, and one or more proximal cooling fluid return tubes 114. As described herein, generator 102 is generally a radiofrequency (RF) generator; however, generator 102 may be another type of energy source, such as microwave energy, thermal energy, ultrasound, or optical energy. In some implementations, generator 102 includes a display (not shown) that displays various aspects of a treatment procedure, such as any parameters that are relevant to a treatment procedure, for example temperature, impedance, etc., and errors or warnings related to a treatment procedure. Alternatively, generator 102 may include means of transmitting a signal to an external display, as described in greater detail below. Generator 102 is generally operable to communicate with probe assembly 106 and the one or more cooling devices 108. Such communication may be unidirectional or bidirectional depending on the devices used and the procedure performed.

In addition, as shown, a distal region 124 of cable 104 may include a splitter 130 that divides cable 104 into two distal ends 136 such that probe assembly 106 can be connected thereto. A proximal end 128 of cable 104 is connected to generator 102. This connection can be permanent, whereby, for example, the proximal end 128 of cable 104 is embedded within generator 102, or temporary, whereby, for example, the proximal end 128 of cable 104 is connected to generator 102 via an electrical connector. The two distal ends 136 of the cable 104 can terminate in connectors 140 operable to couple to probe assembly 106 and establish an electrical connection between probe assembly 106 and the generator 102. In some implementations, system 100 may include a separate cable for each probe assembly (e.g., where there are multiple of probe assembly 106) to couple each probe assembly to generator 102.

In some implementations, cooling device(s) 108 may include any means of reducing a temperature of material located at and proximate to probe assembly 106. For example, cooling devices 108 may include a pump assembly, such as a bidirectional pump assembly, operable to circulate a fluid from the cooling devices 108 through one or more proximal cooling fluid supply tubes 112, probe assembly 106 (e.g., through an internal cavity of probe assembly 106), one or more proximal cooling fluid return tubes 114, and back to cooling devices 108.

In some implementations, proximal cooling fluid supply tubes 112 may include proximal supply tube connectors 116 at the distal ends of proximal cooling fluid supply tubes 112. Additionally, proximal cooling fluid return tubes 114 may include proximal return tube connectors 118 at the distal ends of proximal cooling fluid return tubes 114. In some implementations, proximal supply tube connectors 116 are female Luer-lock type connectors and proximal return tube connectors 118 are male Luer-lock type connectors; although, other connector types are intended to be within the scope of the present disclosure.

Still referring to FIG. 1, probe assembly 106 is shown to include a proximal region 160, a handle 180, a hollow elongate shaft 184, which can also be referred to as an electrocap, and a distal tip region 190 that includes the one or more energy delivery devices 192 and that can also be referred to as the active tip. In some implementations, elongate shaft 184 is manufactured out of polyimide, which provides electrical insulation while maintaining sufficient flexibility and compactness. In other implementations, elongate shaft 184 is any other material imparting similar qualities. In yet other implementations, the elongate shaft 184 is manufactured from an electrically conductive material and may be covered by an insulating material so that delivered energy remains concentrated at energy delivery device 192 of distal tip region 190. Proximal region 160 generally includes a distal cooling fluid supply tube 162, a distal supply tube connector 166, a distal cooling fluid return tube 164, a distal return tube connector 168, a probe assembly cable 170, and a probe cable connector 172. In other implementations, distal cooling fluid supply tube 162 and distal cooling fluid return tube 164 are flexible to allow for greater maneuverability of probe assembly 106, but alternate embodiments with rigid tubes are possible.

In some implementations, distal supply tube connector 166 may be a male Luer-lock type connector and distal return tube connector 168 may be a female Luer-lock type connector. Thus, proximal supply tube connector 116 may be operable to interlock with distal supply tube connector 166 and proximal return tube connector 118 may be operable to interlock with distal return tube connector 168.

In some implementations, probe cable connector 172 may be located at a proximal end of probe assembly cable 170 and may be operable to reversibly couple to one of connectors 140, thus establishing an electrical connection between the generator 102 and probe assembly 106. In some implementations, probe assembly cable 170 includes one or more conductors to transmit RF current from the generator 102 to the one or more energy delivery devices 192, as well as to connect multiple temperature sensing devices to generator 102 as discussed below.

Generally, energy delivery devices 192 may include any means of delivering energy to a region of tissue adjacent to distal tip region 190. For example, energy delivery devices 192 may include an ultrasonic device, an electrode or any other energy delivery means and the invention is not limited in this regard. Similarly, energy delivered via energy delivery devices 192 may take several forms including but not limited to thermal energy, ultrasonic energy, radiofrequency (RF) energy, microwave energy or any other form of energy; however, in CRFA implementations, energy delivery devices 192 is generally configured to deliver RF energy. In some implementations, energy delivery devices 192 includes one or more electrodes for delivering said RF energy. The active region of the electrode may be between 2 and 20 millimeters (mm) in length and energy delivered by the electrode is electrical energy in the form of current in the RF range; however, it should be appreciated that the present disclosure is not intended to be limited in this regard. Different sizes of active regions, all of which are within the scope of the present disclosure, may be used depending on the specific procedure being performed. In some implementations, feedback from generator 102 may automatically adjust the exposed area of energy delivery device 192 in response to a given measurement such as impedance or temperature. For example, energy delivery devices 192 may maximize energy delivered to the tissue by implementing at least one additional feedback control, such as a rising impedance value.

As shown, system 100 may include a controller 120, which may be a separate component from generator 102 (e.g., as shown in FIG. 1) or which may be included in generator 102. For example, while not illustrated, controller 120 or the functionality thereof, as described herein, may be incorporated into generator 102 via suitable computing components. In some such implementations, controller 120 does not separately communicate with generator 102 but may communicate internally with various other components of generator 102 (e.g., via a communications bus). In implementations where controller 120 is separate from generator 102, controller 120 may communicate with generator 102 via any suitable wired or wireless techniques.

In some implementations, controller 120-independently or through generator 102—receives data and measurements from the various components of system 100. In some implementations, for example, controller 120 receives temperature measurements from probe assembly 106. Based on the temperature measurements, generator 102 may perform some action, such as modulating the power that is sent to probe assembly 106, e.g., based on control signals sent by controller 120. As another example, the pumps associated with the cooling devices 108 may communicate a fluid flow rate to controller 120 and may receive control signals from generator 102 or controller 120 instructing the pumps to modulate this flow rate. In other words, controller 120—alone or in combination with the features of generator 102—can control operations of system 100.

### Patient-Specific CRFA Parameters

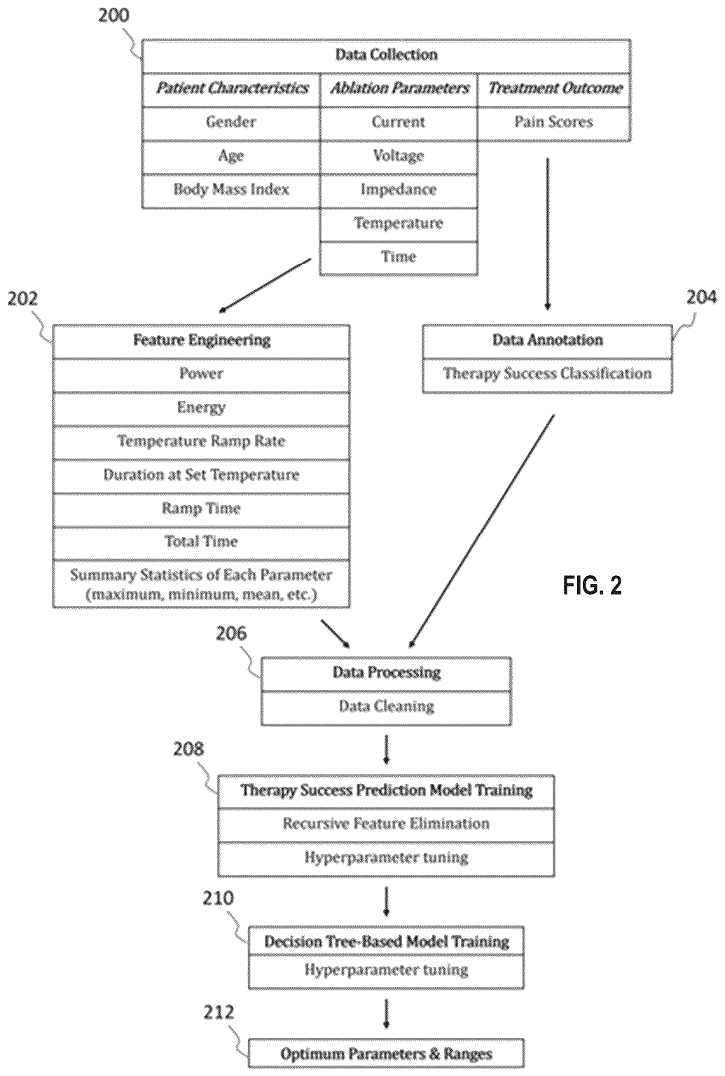

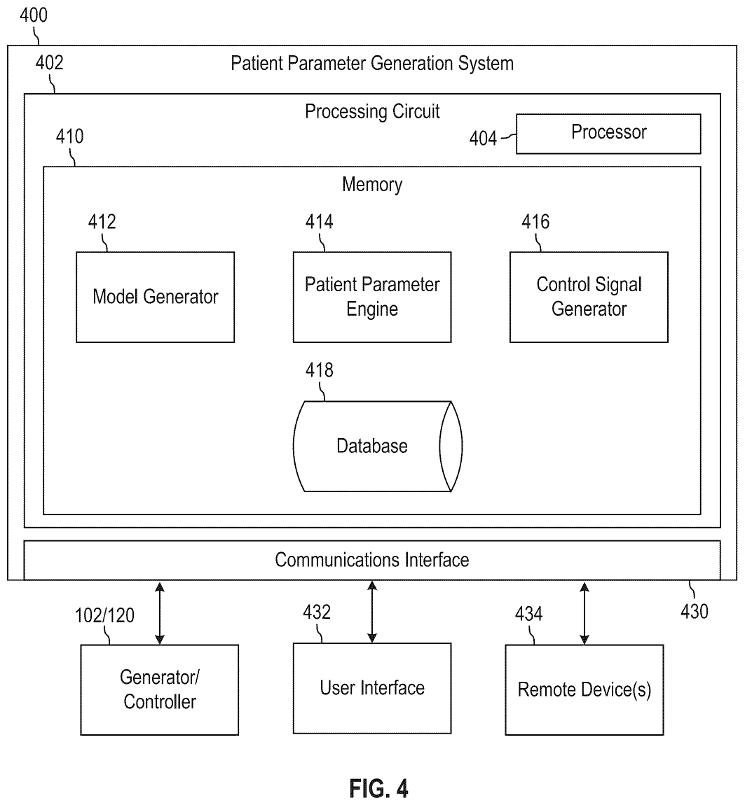

Referring now to FIG. 2, an overview of a process for generating patient-specific CRFA parameters is shown, according to some implementation. As described herein, “patient-specific CRFA parameters” refer to operating parameters and/or settings of a CRFA system (e.g., system 100) that are optimized for a specific patient and/or group of patients. As mentioned above, the patient-specific CRFA parameters that are generated can then be used to control a CRFA system (e.g., system 100) during a procedure. Accordingly, it will be appreciated that the system and/or methods described below may be incorporated with, or otherwise implemented in conjunction with, system 100, as described above. For example, as described below with respect to FIG. 4, controller 120 and/or a separate computing device may be configured to perform the various processes described herein (e.g., the process illustrated in FIG. 2).

Generating patient-specific CRFA parameters, as described herein, generally begins with data collection 200, which can include obtaining historical data for patients that have undergone CRFA procedure. Specifically, historical data may include patient characteristics along with ablation parameters (also referred to herein as “operating parameters” for a CRFA system) and treatment outcomes from each patient's respective CRFA procedure. As shown in FIG. 2, patient characteristics may include, but are not limited to, age, gender, body mass index (BMI), and the like. Ablation parameters can include, but are not limited to, current, voltage, impedance, temperature, time, and the like. Treatment outcomes can be denoted as pain scores, e.g., which may be reported by a patient after their CRFA procedure, such as NRS, GPE, EQ-5D-5L, etc.

As shown, in some implementations, the “raw” historical CRFA data that is collected (e.g., at data collection 200) is further processed, e.g., using various feature engineering and/or data annotation techniques—illustrated as feature engineering 202 and data annotation 204. Feature engineering generally refers to transforming and/or otherwise manipulating the historical patient characteristic and/or operating parameter data to extract features that can be used for training an ensemble machine learning model. For example, featuring engineer 202 shows that the operating parameters included in the historical CRFA data can be used to determine additional features such as power, energy, and rates, along with summary statistics of each parameter (e.g., maximum, minimum, mean, and median values). At data annotation 204, the treatment outcomes may be normalized and/or transformed into targets of binary classification according to the definition of therapy success for the NRS pain score.

After feature engineering 202 and data annotation 204, the historical data can undergo additional processing, e.g., to clean the data—shown as data processing 206. Data cleaning can include, for example, removing or imputing missing data. After, the processed data may be used to train an ensemble machine learning model to predict therapy success, as shown at training step 208. Generally, the ensemble machine learning model described herein includes bagged and/or gradient boosted decision trees and artificial neural networks (ANNs). The ensemble machine learning model is, in particular, trained to identify features of importance, which are operating parameters for a CRFA system that have a significant impact on the likelihood of success of a CRFA procedure. Specifically, the features of importance may be a subset of the CRFA system operating parameters that most greatly improve the likelihood of success of a CRFA procedure. In some such implementations, as shown, the identification of features of importance, model types, and their hyperparameters are based on the best performance of a final model. For example, model performance can be evaluated by determining the area under the curve (AUC), accuracy, or other parameter(s) that indicate how balanced the classes of the ensemble machine learning model are.

After training the ensemble machine learning model some implementations, a decision tree-based model, e.g., a gradient boosted decision tree, is then implemented to find optimized parameters and/or parameters ranges for each feature of importance based on binary classification of therapy success. The decision tree-based model may find optimized parameters and/or parameters ranges by minimizing a loss function based on error between prediction and truth. More generally, the decision tree-based model may solve for an objective function to identify optimized parameters and/or parameters ranges. In some implementations, the decision tree can be pruned to limit to just a few critically important parameters and to minimize overfitting.

In some implementations, when new data is collected (e.g., as new patients undergo CRFA), the above-discussed steps may be repeated to update the final CRFA parameter settings. In other words, the ensemble machine learning model may be retrained and/or re-executed to include additional data, to improve results. Furthermore, this technique can be applied to other anatomical sites that are indicated for CRFA. As noted above, system 100 may further operated/controlled (e.g., to include current adjustment and temperature adjustment) based on the determined CRFA parameter settings. For example, various inputs can be modified during the application of the CRFA energy to modify the output to adjust for patient-specific uses. Machine learning techniques, as described herein, can be used to analyze and adapt the ablation settings to improve the probability of successful therapy success. Such parameters or settings of various aspects of the RF ablation device may include current, voltage, impedance, power, temperature, etc., which may be used to train machine learning models.

As discussed herein, predictions and performance of a machine learning model are based on weights and metrics computed from a training data set provided and are influenced by model types, their respective hyperparameters, and method to pool ensemble member predictions. The training data set can be updated and the machine learning model recomputed with hyperparameter tuning in accordance with principles of machine learning such that updated clinical data can be used to update the machine learning model to provide update settings for the application of the CRFA to a patient, where settings can use various aspects of the condition or patient to tailor the settings of the device for a particular instance of use of the CRFA device.

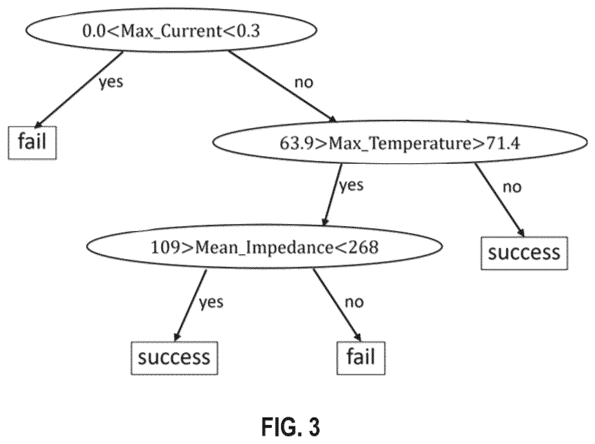

Turning briefly to FIG. 3, a diagram of an example decision tree for predicting CRFA therapy success is shown, according to some implementations. As shown, the decision tree includes identified parameters of importance for CRFA and their corresponding parameter ranges for therapy success. In some implementations, the tree is pruned to reduce risk of overfitting as well as for simplicity and is computed using clinical CRFA data on the knee with NRS pain scores. According to the tree in this example, there is higher probability of therapy success if maximum-current above 0.3 A (i.e., sufficient current is delivered to ablate nerve tissue) and maximum-temperature between 63.9 to 71.4° F. (i.e., tissue has reached high enough temperature to cause temporary nerve damage), and if mean-impedance is between 109 and 468 Q (i.e., no permanent desiccation of tissue due to excessive heat) as well. Using these CRFA settings may lead to more consistent and greater therapy outcome success.

Using preliminary pain score data from clinical CRFA studies of the knee, a pruned decision tree was found to have accuracy of 77.6% and AUC of 77.9% for predicting therapy success. It should be understood, however, that the system and method of the present disclosure may be further applied towards other CRFA involving any anatomy structure indicated for CRFA in addition to those relating to the knee. Additionally, the system and method of the present disclosure is not limited just to NRS and may be further applied towards other pain scores like GPE (global perceived effect), OA grade, EQ-5D-5L, KOOS Jr, and WOMAC, along with each of their respective definitions of therapy success.

### Patient Parameter Generation System

Referring now to FIG. 4, a block diagram of patient parameter generation system 400 is shown, according to some implementations. As described herein, system 400 may be a stand-alone system or may be implemented via a device that is separate from system 100. Specifically, system 400 may be separate from generator 102 and/or controller 120. In some such implementations, it should be appreciated that system 400 may communicate with generator 102 and/or controller 120, as described below, to implement the various processes and features described herein. In some implementations, system 400 is a part of generator 102 and/or controller 120. For example, system 400 and the features thereof may be implemented by controller 120. Thus, it should be appreciated that the following description of system 400 is not intended to be limiting in this regard.

System 400 is shown to include a processing circuit 402 that includes a processor 404 and a memory 410. Processor 404 can be a general-purpose processor, an ASIC, one or more FPGAs, a group of processing components, or other suitable electronic processing structures. In some embodiments, processor 404 is configured to execute program code stored on memory 410 to cause system 400 to perform one or more operations, as described below in greater detail. It will be appreciated that, in embodiments where system 400 is part of another computing device, the components of system 400 may be shared with, or the same as, the host device. For example, if system 400 is implemented via generator 102, then system 400 may utilize the processing circuit, processor(s), and/or memory of generator 102 to perform the functions described herein, or vice versa.

Memory 410 can include one or more devices (e.g., memory units, memory devices, storage devices, etc.) for storing data and/or computer code for completing and/or facilitating the various processes described in the present disclosure. In some embodiments, memory 410 includes tangible (e.g., non-transitory), computer-readable media that stores code or instructions executable by processor 404. Tangible, computer-readable media refers to any physical media that is capable of providing data that causes system 400 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 410 can include RAM, ROM, hard drive storage, temporary storage, non-volatile memory, flash memory, optical memory, or any other suitable memory for storing software objects and/or computer instructions. Memory 410 can include database components, object code components, script components, or any other type of information structure for supporting the various activities and information structures described in the present disclosure. Memory 410 can be communicably connected to processor 404, such as via processing circuit 402, and can include computer code for executing (e.g., by processor 404) one or more processes described herein.

While shown as individual components, it will be appreciated that processor 404 and/or memory 410 can be implemented using a variety of different types and quantities of processors and memory. For example, processor 404 may represent a single processing device or multiple processing devices. Similarly, memory 410 may represent a single memory device or multiple memory devices. Additionally, in some embodiments, system 400 may be implemented within a single computing device (e.g., one server, one housing, etc.). In other embodiments, system 400 may be distributed across multiple servers or computers (e.g., that can exist in distributed locations). For example, system 400 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. For example, virtualization software may be employed by system 400 to provide the functionality of a number of servers that is not directly bound to the number of computers in system 400.

Memory 410 is shown to include a model generator 412 configured to generate and/or train a predictive model (or multiple different predictive models) to predict a likelihood of success of a CRFA treatment procedure, which is turn is used to identify feature of importance (e.g., ablation parameters) that affect the likelihood of success of a CRFA treatment procedure. The “predictive model,” in this case, refers to an ensemble machine learning model, as mentioned above. To this point, model generator 412 may be configured to generate and/or train an ensemble machine learning model that can predict ablation parameters (e.g., current, voltage, temperature, time, etc.) based on patient characteristics (e.g., age, gender, BMI, etc.) that are most important to affecting the success of an ablation procedure. As mentioned above, the ensemble machine learning model that is generated and/or trained by model generator 412 may include one or more decision trees and/or one or more ANNs.

As also mentioned above, training an ensemble machine learning model to identify treatment parameters of importance generally begins with obtaining historical data from a plurality of previously performed CRFA procedures. In some such implementations, model generator 412 may obtain this sort of historical data from a database (e.g., database 418), via a user input, or by any other suitable method. As mentioned above, the historical data may include patient characteristics, ablation parameters, and procedure outcomes. In some implementations, the historical data may be engineered, augmented, cleaned, annotated, or otherwise preprocessed before being used to train ensemble machine learning model. The machine learning model-part of model generator 412-may be used to generate a list of “feature of importance” that are indicative/predictive of a likelihood of success of a CRFA treatment procedure, include patient characteristics and ablation settings that most impact success.

Optimized parameters and/or parameters ranges for each feature of importance can be determined using a decision tree-based model. In some implementations, the decision tree is a gradient boosted decision tree. In some implementations, model generator 412 can prune the decision tree to further narrow the results and prevent overfitting. In some implementations, after generation, model generator 412 is configured to maintain a machine learning model that includes data related to the application of CRFA including treatment outcomes, patient characteristics, and ablation parameters. In some implementations, model generator 412 recomputes the machine learning model based on updated data relating to treatment outcomes, patient characteristics, and/or ablation parameters.

Memory 410 is also shown to include a patient parameter engine 414 configured to generate patient-specific parameters based on input patient characteristics. In particular, patient parameter engine 414 is generally configured to apply the predictive model generated/trained by model generator 412 to determine patient-specific parameters for additional patients. In some implementations, patient parameter engine 414 receives patient characteristics, e.g., via a user input, and provides the patient characteristics as inputs to the predictive model. In return, the predictive model generally outputs ablation parameters that have been optimized to the patient.

Memory 410 is also shown to include a control signal generator 416 configured to generate and transmit control signals. Specifically, control signal generator 416 can generate signals for controlling the various components of system 100, such as generator 102. For example, control signal generator 416 may transmit control signals to generator 102, which in turn controls the energy output through probe assembly 106. Thus, in some implementations, the “control signals” described herein include data which command generator 102 and/or controller 120 to operate system 100 and the components thereof according to specific parameters. For example, control signal generator 416 may transmit ablation parameters to generator 102 for controlling operations of system 100.

System 400 is also shown to include a communications interface 430 that facilitates communications between (e.g., transmitting data to and/or receiving data from) and any external components or devices, such as generator 102 and/or controller 120. Accordingly, communications interface 430 can be or can include a wired or wireless communications interface (e.g., jacks, antennas, transmitters, receivers, transceivers, wire terminals, etc.) for conducting data communications, or a combination of wired and wireless communication interfaces. In some embodiments, communications via communications interface 430 are direct (e.g., local wired or wireless communications) or via a network (e.g., a WAN, the Internet, a cellular network, etc.). For example, communications interface 430 may include one or more Ethernet ports for communicably coupling system 400 to a network (e.g., the Internet). In another example, communications interface 430 can include a Wi-Fi transceiver for communicating via a wireless communications network. In yet another example, communications interface 430 may include cellular or mobile phone communications transceivers.

In some implementations, system 400 is communicably coupled to a user interface 432, e.g., via communications interface 430. Generally, user interface 432 is and/or includes any electronic device that is configured to display information in the form of text, graphics, pictures, video, and the like. User interface 432 may include an LED or LCD display, for example. In particular, user interface 432 may be configured to display information related to system 400, such as patient characteristics, ablation parameters, and the like. In some implementations, user interface 432 includes one or more user input devices, such as a mouse, a keyboard, a number pad, buttons, etc. In some implementations, user interface 432 includes a touch screen which incorporated both display and user input functionalities. In some implementations, user interface 432 includes one or more display and/or user input components that are positioned on generator 102 and/or controller 120. For example, user interface 432 can be a user interface positioned on controller 120 to display current and set ablation parameters.

In some implementations, system 400 is communicably coupled to remote device(s) 434 via communications interface 430 (e.g., via a wireless network). Remote device(s) 434 may be any computing devices—typically including a memory (e.g., RAM, ROM, Flash memory, hard disk storage, etc.), a processor (e.g., a general-purpose processor, an application specific integrated circuit (ASIC), one or more field programmable gate arrays (FPGAs), a group of processing components, or other suitable electronic processing components), and optionally a user interface (e.g., a touch screen)—that provide remote access to system 400. Remote device(s) 434 can include, for example, mobile phones, electronic tablets, laptops, desktop computers, workstations, and other types of electronic devices. For example, remote device(s) 434 may include one or more computers and/or one or more servers, e.g., for storing and/or manipulating data.

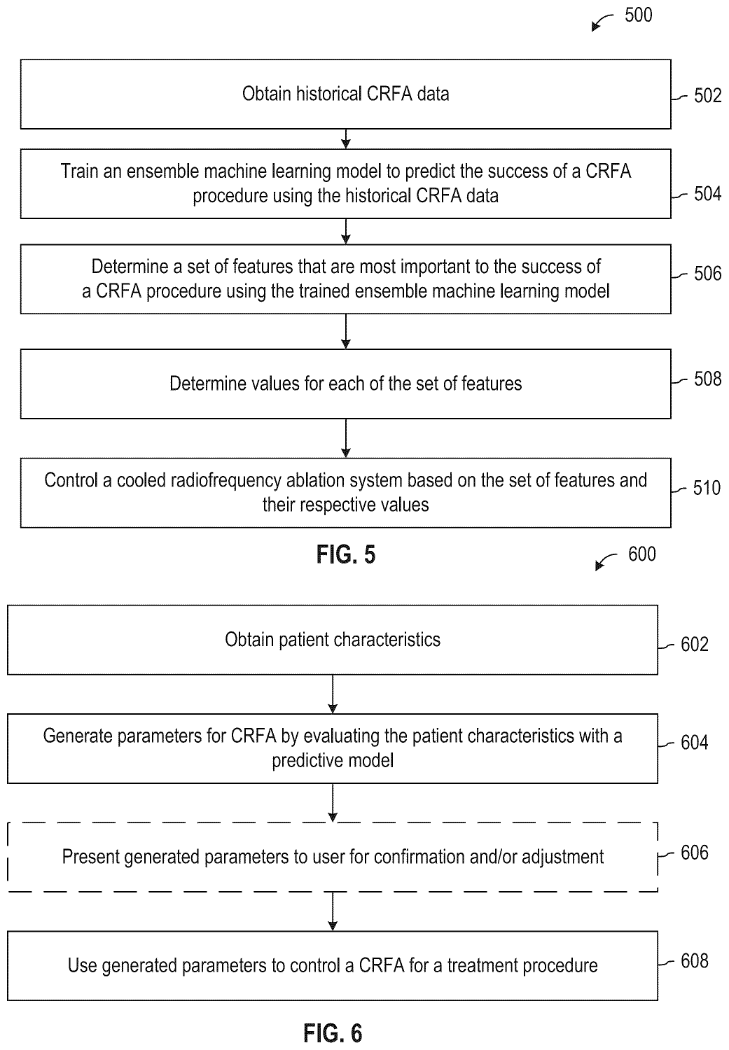

Referring now to FIG. 5, a flow diagram of a process 500 for developing a predictive model for determining patient-specific CRFA parameters is shown, according to some implementations. In some implementations, process 500 is implemented by system 400, as described above. Accordingly, in some implementations, process 500 may more broadly be implemented by generator 102 and/or controller 120. In some implementations, process 500 is cooperatively performed by two or more of generator 102, controller 120, and system 400. It will be appreciated that certain steps of process 500 may be optional and, in some implementations, process 500 may be implemented using less than all of the steps. It will also be appreciated that the order of steps shown in FIG. 5 is not intended to be limiting.

At step 502, patient characteristics and ablation parameters are obtained. Specifically, historical CRFA data that includes patient characteristics and corresponding ablation parameters associated with a plurality of patients previously treated using CRFA are obtained. In some implementations, the historical data further includes treatment outcomes for each patient. In some implementations, prior to step 502, the historical CRFA data is collected, e.g., over a period of time. In some implementations, a dataset of features (e.g., patient characteristics and ablation data) and target (e.g., therapy outcome) is divided into a training and a validation dataset. In some implementations, historical ablation data includes one of more of current, voltage, impedance, power, temperature, duration, total treatment time, temperature ramp rate, and ramp time, and the like. In some implementations, historical patient characteristics include patient age, gender, BMI, and the like. In some implementations, the treatment outcomes are based on standard pain scores. In some implementations, the pain scores are normalized by max-min scaling to the baseline pain level.

At step 504, the success of a CRFA treatment is predicted using ensemble machine learning. In some implementations, ensemble machine learning includes one or more bagged and gradient boosted decision trees and/or one or more neural networks. In some implementations, after feature engineering and data annotation, the data undergoes processing where missing features are cleaned either by removal or imputation. Once prepared, the data is used to identify features of importance to predict therapy success by training for an ensemble model, consisting of bagged and gradient boosted decision trees and neural networks in this example. In some implementations, selection of features of importance, model types and their hyperparameters are based on the best performance of the final model.

At step 506, a list of important features is generated. Generally, the list of important features is generated by identifying the features from that are most relevant to success using the ensemble machine learning model at step 504. In some implementations, the list of important features is generated by performing recursive feature elimination and cross validation on an ensemble model consisting of bagged and gradient boosted decision trees and neural networks. In some implementations, the list of important features is generated by collecting ablation data of the electrical energy delivered during the CRFA procedure, patient characteristics, and patient reported pain scores. Then, therapy outcomes can be derived based on pain scores. In some implementations, recursive feature elimination and cross validation is performed on the training dataset (e.g., from step 502) to find list of important features that contribute most to prediction of therapy outcome. In some implementations, the validation dataset is utilized to evaluate the tuned hyperparameters to the ensemble model.

At step 508, the list of important features is narrowed using gradient boosted decision tree. In some implementations, the narrowed list of features define the settings for CRFA that are most important to therapy success. In some implementations, gradient boosted decision tree is trained to find optimized parameters ranges to split at each decision node for the binary classification of therapy success by minimizing the loss function based on error between prediction and truth. In some implementations, the decision tree is pruned to reduce model complexity and minimize overfitting.

At step 510, a decision tree of identified features and their corresponding levels/values is generated. In some implementations, this includes computing parameter ranges at each decision or terminal node after splitting for therapy success. In some implementations, identifying levels/values for the identified features includes applying the features to a decision tree to determine the most important ablation parameters and their settings for use in CRFA. In some implementations, new CRFA data can be collected from a plurality of second patients that are treated based on the second set of ablation parameters and their corresponding ranges of values. In some such implementations, the new CRFA data can be used to update the predictive model using hyperparameter. In some such implementations, the machine learning model is recomputed based on the new data.

Referring now to FIG. 6, a flow diagram of a process 600 for determining patient- specific CRFA parameters using a trained predictive model is shown, according to some implementations. In some implementations, process 600 is implemented by system 400, as described above. Accordingly, in some implementations, process 600 may more broadly be implemented by generator 102 and/or controller 120. In some implementations, process 600 is cooperatively performed by two or more of generator 102, controller 120, and system 400. It will be appreciated that certain steps of process 600 may be optional and, in some implementations, process 600 may be implemented using less than all of the steps. It will also be appreciated that the order of steps shown in FIG. 6 is not intended to be limiting.

At step 602, patient characteristics are obtained. In some implementations, patient characteristics are obtained via a user input, e.g., from a physician or other operator of system 100. Generally, the patient characteristics include characteristics that affect ablation success, e.g., as detected via process 500. For example, the patient characteristics can include patient age, gender, BMI, and the like. These types of characteristics can be entered into a user interface, for example, to be provided to system 400, prior to initiating a CRFA procedure.

At step 604, parameters for CRFA are generated by evaluating the patient characteristics with a predictive model. In particular, the patient characteristics obtained at step 602, e.g., via user input, are provided as inputs to the predictive model generated via process 500. The predictive model may then output ablation parameters that have be optimized for the particular patient, based on their characteristics.

At step 606, the generated parameters are optionally presented to a user. For example, the generated parameters may be displayed via a user interface or transmitted to a remote/external device for display. In some implementations, ablation parameters can be calculated directly on generator 102 or controller 120 and are therefore displayed on a user interface of either generator 102 or controller 120 prior to the start of a CRFA procedure. In this manner, a user (e.g., a physician) can view the parameters for confirmation and/or adjustment.

At step 608, the generated parameters are optionally used to control a CRFA for a treatment procedure. In some implementations, the ablation parameters are directed used to control system 100 throughout the procedure. For example, the treatment parameters can be applied to generator 102 and/or controller 120 to control the RF energy output via probe assembly 106. In this way, system 100 can be operated at parameters that have been optimized for a particular patient.

### Configuration of Certain Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

The present disclosure contemplates methods, systems, and program products on any machine-readable media for accomplishing various operations. Certain implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine- executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “and” “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of implementations of the disclosed methods.

Throughout this application, various publications may have been referenced. The disclosures of these publications in their entireties are hereby incorporated by reference into this application in order to more fully describe the state of the art to which this invention pertains.

### Example Implementations

Example 1. A method of determining parameter settings for use in cooled radio frequency ablation, comprising: maintaining a machine learning model comprising a data related to the application of cooled radio frequency ablation including treatment outcomes, patient characteristics, and ablation parameters, the method comprising; identifying a list of features of importance to predict therapy success based on the data, including historical patient characteristics and ablation; applying decision tree to the list of important features to narrow the list of important features; computing parameter ranges at each decision or terminal node after splitting for therapy success; updating the data with updated treatment outcomes, patient characteristics, and ablation parameters; and recomputing the machine learning model. Example 2. The method of example 1, wherein the CRFA data is from the shoulder, knee, hip, lumbar, thoracic, cervical, sacroiliac, and disc.

Example 3. The method of example 1, wherein the historical ablation data are selected from among current, voltage, impedance, power, temperature, duration, total treatment time, temperature ramp rate, and ramp time, and wherein patient characteristics include patient age, gender and body mass index.

Example 4. The method of example 1, wherein the treatment outcomes are based on standard pain scores.

Example 5. The method of example 1, wherein the pain scores are normalized by max-min scaling to the baseline pain level.

Example 6. The method of example 1, wherein the therapy success is defined according to the definition for each pain score.

Example 7. The method of example 1, wherein a list of features of importance to predict therapy outcome is identified by performing recursive feature elimination and cross validation on an ensemble model consisting of bagged and gradient boosted decision trees and neural networks.

Example 8. The method of claim 7, wherein identifying the list of features of importance to predict therapy outcome comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; performing recursive feature elimination and cross validation on the training dataset to find list of important features that contribute most to prediction of therapy outcome; utilizing the validation dataset to evaluate the tuned hyperparameters to the ensemble model.

Example 9. The method of example 1, wherein the identified features of importance are applied to a decision tree to determine the most important ablation parameters and their settings for use in CRFA.

Example 10. The method of example 9, further comprising of a gradient boosted decision tree trained to find the optimum parameters ranges to split at each decision node for the binary classification of therapy success by minimizing the loss function based on error between prediction and truth.

Example 11. The method of example 10, wherein the training of the decision tree to find the optimum parameter ranges for therapy success comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; utilizing the training dataset to train the decision tree model; utilizing the validation dataset to evaluate the tuned hyperparameters to the decision tree model.

Example 12. The method of example 10, further comprising of pruning the decision tree to reduce model complexity and minimize overfitting.

Example 13. A system for determining settings for use in cooled radio frequency ablation, comprising a processor and a memory in communication with the processor and storing thereon computer-readable instructions that, when executed by the processor, cause the processor to: maintain a machine learning model comprising a data related to the application of cooled radio frequency ablation including treatment outcomes, patient characteristics, and ablation parameter; identify a list of features of importance to predict therapy outcome based on the data, including historical patient characteristics and ablation; apply decision tree to the list of important features to narrow the list of important features; compute parameter ranges at each decision or terminal node after splitting for therapy success; update the data with updated treatment outcomes, patient characteristics, and ablation parameters; and recompute the machine learning model.

Example 14. The system of example 13, wherein the CRFA data is from the shoulder, knee, hip, lumbar, thoracic, cervical, sacroiliac, and disc.

Example 15. The system of example 13, wherein the historical ablation data are selected from among current, voltage, impedance, power, temperature, duration, total treatment time, temperature ramp rate, and ramp time. Patient characteristics include patient age, gender and body mass index.

Example 16. The system of example 13, wherein the treatment outcomes are based on standard pain scores.

Example 17. The system of example 13, wherein the pain scores are normalized by max-min scaling to the baseline pain level.

Example 18. The system of example 13, wherein the therapy success is defined according to the definition for each pain score.

Example 19. The system of example 13, wherein a list of features of importance to predict therapy outcome is identified by performing recursive feature elimination and cross validation on an ensemble model consisting of bagged and gradient boosted decision trees and neural networks.

Example 20. The system of example 19, wherein identifying the list of features of importance to predict therapy outcome comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; performing recursive feature elimination and cross validation on the training dataset to find list of important features that contribute most to prediction of therapy outcome; utilizing the validation dataset to evaluate the tuned hyperparameters to the ensemble model.

Example 21. The system of example 13, wherein the identified features of importance are applied to a decision tree to determine the most important ablation parameters and their settings for use in CRFA.

Example 22. The system of example 21, further comprising of a gradient boosted decision tree trained to find the optimum parameters ranges to split at each decision node for the binary classification of therapy success by minimizing the loss function based on error between prediction and truth.

Example 23. The system of example 21, wherein the training of the decision tree to find the optimum parameter ranges for therapy success comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; utilizing the training dataset to train the decision tree model; utilizing the validation dataset to evaluate the tuned hyperparameters to the decision tree model.

Example 24. The system of example 21, further comprising pruning the decision tree to reduce model complexity and minimize overfitting.

Example 25. A non-transitory computer readable medium according to principles described herein may have stored thereon program instructions that, when executed by a processor to determine settings for use in cooled radio frequency ablation cause the processor to: maintain a machine learning model comprising a data related to the application of cooled radio frequency ablation including treatment outcomes, patient characteristics, and ablation parameters; identify a list of features of importance to predict therapy outcome based on the data, including historical patient characteristics and ablation; apply decision tree to the list of important features to narrow the list of important features; compute parameter ranges at each decision or terminal node after splitting for therapy success; update the data with updated treatment outcomes, patient characteristics, and ablation parameters; and recompute the machine learning model.

Example 26. The non-transitory computer-readable medium of example 25, wherein the CRFA data is from the shoulder, knee, hip, lumbar, thoracic, cervical, sacroiliac, and disc.

Example 27. The non-transitory computer-readable medium of example 25, wherein the historical ablation data are selected from among current, voltage, impedance, power, temperature, duration, total treatment time, temperature ramp rate, and ramp time, and wherein patient characteristics include patient age, gender and body mass index.

Example 28. The non-transitory computer-readable medium of example 25, wherein the treatment outcomes are based on standard pain scores.

Example 29. The non-transitory computer-readable medium of example 25, wherein the pain scores are normalized by max-min scaling to the baseline pain level.

Example 30. The non-transitory computer-readable medium of example 25, wherein the therapy success is defined according to the definition for each pain score.

Example 31. The non-transitory computer-readable medium of example 25, wherein a list of features of importance to predict therapy outcome is identified by performing recursive feature elimination and cross validation on an ensemble model consisting of bagged and gradient boosted decision trees and neural networks.

Example 32. The non-transitory computer-readable medium of example 31, wherein identifying the list of features of importance to predict therapy outcome comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; performing recursive feature elimination and cross validation on the training dataset to find list of important features that contribute most to prediction of therapy outcome; utilizing the validation dataset to evaluate the tuned hyperparameters to the ensemble model.

Example 33. The non-transitory computer-readable medium of example 25, wherein the identified features of importance are applied to a decision tree to determine the most important ablation parameters and their settings for use in CRFA.

Example 34. The non-transitory computer-readable medium of example 33, further comprising of a gradient boosted decision tree trained to find the optimum parameters ranges to split at each decision node for the binary classification of therapy success by minimizing the loss function based on error between prediction and truth.

Example 35. The non-transitory computer-readable medium of example 33, wherein the training of the decision tree to find the optimum parameter ranges for therapy success comprises: collecting ablation data of the electrical energy delivered during the CRFA procedure, the patient characteristics and patient reported pain scores; deriving the therapy outcomes as defined based on pain scores; dividing the dataset of features (patient characteristics and ablation data) and target (therapy outcome) into a training and a validation dataset; utilizing the training dataset to train the decision tree model; utilizing the validation dataset to evaluate the tuned hyperparameters to the decision tree model.

Example 36. The non-transitory computer-readable medium of example 33, further comprising of pruning the decision tree to reduce model complexity and minimize overfitting.

What is claimed is:

1. A method for determining settings for cooled radiofrequency ablation (CRFA) system, the method comprising:
obtaining historical CRFA data associated with a plurality of patients previously treated using the CRFA system, wherein the historical CRFA data includes patient characteristics, operating parameters of the CRFA system, and treatment outcomes associated with each of the plurality of patients;
training an ensemble machine learning model to predict success of CRFA procedures based on the historical CRFA data;
determining a first set of operating parameters for the CRFA system using the trained ensemble machine learning model, wherein the first set of operating parameters comprise settings of the CRFA system that are determined to affect the success of CRFA procedures; and
determining a value or a range of values for each of the first set of operating parameters for use in CRFA treatment procedures using a decision tree-based model.

2. The method of claim 1, further comprising presenting the first set of operating parameters, and each corresponding value or range of values, via a user interface.

3. The method of claim 1, further comprising operating the CRFA ablation system based on the first set of operating parameters, and each corresponding value or range of values.

4. The method of claim 1, further comprising cleaning and/or annotating the historical CRFA data prior to training the ensemble machine learning model, wherein cleaning comprises removing or imputing missing data.

5. The method of claim 1, further comprising:
collecting additional CRFA data from a plurality of second patients that are treated based on the first set of operating parameters, and each corresponding value or range of values; and
retraining the ensemble machine learning model using hyperparameter tuning based on the additional CRFA data.

6. The method of claim 1, further comprising:
receiving new patient characteristics for a new patient that is set to undergo a CRFA treatment procedure; and
predicting a value or range of values for each of the first set of operating parameters for the CRFA treatment procedure based on the new patient characteristics.

7. The method of claim 1, wherein the ensemble machine learning model comprises one or more bagged and gradient boosted decision trees and/or one or more artificial neural networks.

8. The method of claim 1, further comprising collecting the historical CRFA data by storing the patient characteristics, the operating parameters, and the treatment outcomes associated with the plurality of patients over a period of time.

9. The method of claim 1, wherein the first set of operating parameters comprise one or more of: a current, a voltage, an impedance, a power, a temperature, a treatment duration, a total treatment time, a temperature ramp rate, or a ramp time.

10. The method of claim 1, wherein the operating parameters included in the historical CRFA data comprise one or more of: a current, a voltage, an impedance, a power, a temperature, a treatment duration, a total treatment time, a temperature ramp rate, or a ramp time.

11. The method of claim 1, wherein the patient characteristics comprise one or more of age, gender, or body mass index (BMI).

12. The method of claim 1, wherein the treatment outcomes included in the historical CRFA data are derived from patient-provided pain scores.

13. A system for determining settings for use in cooled radiofrequency ablation (CRFA), the system comprising:
one or more processors; and
memory having instructions stored thereon that, when executed by the one or more processors, cause the system to:
obtain historical CRFA data associated with a plurality of patients previously treated using a CRFA system, wherein the historical CRFA data includes patient characteristics, operating parameters of the CRFA system, and treatment outcomes associated with each of the plurality of patients;
train an ensemble machine learning model to predict success of CRFA procedures based on the historical CRFA data;
determine a first set of operating parameters for the CRFA system using the trained ensemble machine learning model, wherein the first set of operating parameters comprise settings of the CRFA system that are determined to affect the success of CRFA procedures; and
determine a value or a range of values for each of the first set of operating parameters for use in CRFA treatment procedures using a decision tree-based model.

14. The system of claim 13, wherein the instructions further cause the system to:
present the first set of operating parameters, and each corresponding value or range of values, via a user interface.

15. The system of claim 13, wherein the instructions further cause the system to:
control the CRFA ablation system based on the first set of operating parameters, and each corresponding value or range of values.

16. The system of claim 13, wherein the instructions further cause the system to:
the historical CRFA data prior to training the ensemble machine learning model, wherein cleaning comprises removing or imputing missing data.

17. The system of claim 13, wherein the instructions further cause the system to:
collect additional CRFA data from a plurality of second patients that are treated based on the first set of ablation parameters, and each corresponding value or range of values; and
retrain the ensemble machine learning model using hyperparameter tuning based on the additional CRFA data.

18. The system of claim 14, wherein the instructions further cause the system to:
receive new patient characteristics for a new patient that is set to undergo a CRFA treatment procedure; and
predict a value or range of values for each of the first set of operating parameters for the CRFA treatment procedure based on the new patient characteristics.

19. The system of claim 13, wherein the ensemble machine learning model comprises one or more bagged and gradient boosted decision trees and/or one or more artificial neural networks.

20. A non-transitory computer readable medium having instructions stored thereon that, when executed by one or more processors, cause a device to:
obtain historical CRFA data associated with a plurality of patients previously treated using a CRFA system, wherein the historical CRFA data includes patient characteristics, operating parameters of the CRFA system, and treatment outcomes associated with each of the plurality of patients;
train an ensemble machine learning model to predict success of CRFA procedures based on the historical CRFA data;
determine a first set of operating parameters for the CRFA system using the trained ensemble machine learning model, wherein the first set of operating parameters comprise settings of the CRFA system that are determined to affect the success of CRFA procedures; and
determine a value or a range of values for each of the first set of operating parameters for use in CRFA treatment procedures using a decision tree-based model.
